(19) United States
(12) Patent Application Publication
(10) Pub. No.: US 2026/0224911 A1
(43) Pub. Date: Aug. 6, 2026

(54) PHOTOBIOMODULATION AND ELECTRICAL MODULATION OF A TARGET AREA OF THE BRAIN THROUGH AN ELECTRO-OPTICAL CRANIAL WINDOW

(71) Applicant: Case Western Reserve University, Cleveland, OH (US)

(72) Inventors: Michael MOFFITT, Cleveland, OH (US); Michael JENKINS, Cleveland, OH (US)

(21) Appl. No.: 19/154,442

(22) PCT Filed: Feb. 9, 2024
(86) PCT No.: PCT/US2024/015203

§ 371 Date: Aug. 7, 2025

Related U.S. Application Data
(60) Provisional application No. 63/444,356, filed on Feb. 9, 2023.
(60) Provisional application No. 63/604,206, filed on Nov. 30, 2023.

(51) Int. Cl. A61N 5/06; A61N 1/05; A61N 1/36

(52) U.S. Cl. CPC A61N 5/0622; A61N 1/0529; A61N 1/3614; A61N 2005/0626; A61N 2005/0647; A61N 2005/0664

(57) ABSTRACT

A system to treat a target area of a patient's brain can include at least one external electro-optical applicator and at least one window spanning beneath skin covering a skull of the patient and through obstructive material of the skull of the patient. Each electro-optical applicator can include at least one light source and at least one electrical source. Each window can include at least one electrode proximal the target area and a conductive path to the at least one electrode. The at least one window can be configured to: transmit a light signal from the at least one light source through the window to the target area of the brain of the patient; and deliver an electrical modulation from the at least one electrical source via the at least one electrode to the target area.

### CROSS-REFERENCE TO RELATED APPLICATIONS

This application claims the benefit of U.S. Provisional Application No. 63/604,206, filed on 30 Nov. 2023, entitled PBM AND ELECTRICAL MODULATION OF THE PRECUNEUS TO TREAT DIMENTIA. This application also claims the benefit of U.S. Provisional Application No. 63/444,356, filed 9 Feb. 2023, entitled SYSTEMS AND METHODS FOR PHOTOBIOMODULATION OF TARGET TISSUE THROUGH A WINDOW. These provisional applications are hereby incorporated by reference in their entirety for all purposes.

### TECHNICAL FIELD

This disclosure relates generally to treatment of neurological disorders and more specifically to systems and methods that use one or more electro-optical cranial windows (also referred to as “windows”) through a skull of a patient to deliver one or more of photobiomodulation (PBM) and electrical stimulation to a target area within a brain of a patient to treat one or more neurological disorders.

### BACKGROUND

The Default Mode Network (DMN) is a network of brain regions, including the dorsal medial prefrontal cortex, posterior cingulate cortex, precuneus, and angular gyrus, that are active during periods when a person is awake but not under focused cognitive load and fully aware of the outside world. Disruption of the DMN is thought to be related to numerous pathologies, including Alzheimer's disease, autism, schizophrenia, major depressive disorder (MDD), chronic pain, post-traumatic stress disorder (PTSD), attention deficit hyperactivity disorder (ADHD), and the like. The DMN can be modulated with various levels of success using non-invasive techniques, such as pharmaceuticals, exercise, sleep, and the like. A more invasive treatment, deep brain stimulation (DBS), has also been used to modulate the DBN via electrical stimulation and transcranial magnetic stimulation (TMS) has also been used in clinical settings to modulate the DMN. However, deep brain stimulation is extremely invasive, dangerous, and not effective for every patient and TMS requires clinical visits and cannot be used at home. External applications of electrical stimulation and/or photobiomodulation (PBM) have been tested in lieu of DBS, but attenuation due to obstructive materials has been found to significantly lessens the efficacy of the treatments and unwanted side effects have occurred.

### SUMMARY

Described herein are systems and methods that can deliver one or more of photobiomodulation (PBM) and electrical stimulation to a target area of a brain through obstructive material of a patient's skull. One or more electro-optical cranial windows (also referred to as windows) can be configured to deliver the one or more of photobiomodulation (PBM) and electrical stimulation to the target area. The one or more windows are positioned through obstructive material of the patient's skull (e.g., bone, dura, etc.) between skin of the patient and the target area. The one or more windows can deliver a PBM light signal from an external light source to the target area in a safer manner and with significantly less attenuation due to the obstructive material than traditional PBM application. The one or more windows can alternatively and/or additionally transmit an electrical signal to the target area using an electrode not implanted into brain tissue for less invasive electrical stimulation.

In an aspect, the present disclosure can include a system that can deliver one or more of PBM and surface electrical modulation from an external device to a target area within a brain of a patient through a window (also referred to as a cranial window). The system can include at least one electro-optical applicator external to the patient. Each electro-optical applicator can include at least one light source, and at least one electrical source. The system can also include at least one window (e.g., a cranial window) spanning beneath skin covering a skull of the patient and through obstructive material of the skull of the patient. Each window can include at least one electrode proximal a target area of a brain of the patient and a conductive path to the at least one electrode. Each window can also be configured to transmit a light signal from the at least one light source through the window to the target area of the brain of the patient; and deliver an electrical modulation from the at least one electrical source via the at least one electrode to the target area of the brain of the patient.

In a further aspect, the present disclosure can include a system that can deliver one or more of PBM and surface electrical modulation from an external device to a target area within a brain of a patient through a plurality of windows (also referred to as cranial windows). The system includes a first window that spans through obstructive material of a skull of a patient between the skin and a precuneus of a hemisphere of a brain of the patient. The first window includes a first electrode proximal the precuneus of the hemisphere to deliver an electrical stimulation, and a conductive pathway from a top of the window to the first electrode. The system also includes a first electro-optical applicator that includes a light source and an electrical source and configured to align the light source with the first window to deliver a light signal through the first window to the precuneus of the hemisphere and the electrical source with the conductive pathway. The system also includes a second window that spans through obstructive material of the skull of the patient between the skin and another precuneus of another hemisphere of the brain of the patient and includes a second electrode proximal the precuneus of the hemisphere to deliver an electrical stimulation, and another conductive pathway from a top of the window to the first electrode. The system also includes a second electro-optical applicator that includes another light source and another electrical source configured to align the other light source with the second window to deliver another light signal through the second window to the precuneus of the other hemisphere and the other electrical source with the other conductive pathway.

Also described herein are methods for treating one or more pathologies of the Default Mode Network (DMN) using PBM and/or surface electrical modulation of a cranial target area. A window (also referred to as a cranial window) can span through obstructive material of a skull of a patient between the skin and the cortical area of the brain to deliver one or more of photobiomodulation (PBM) and surface electrical modulation from an external device to a target area within a cortical area of the brain of a patient.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a block diagram showing a system that can deliver a light signal and/or an electrical signal transcutaneously through an electro-optical cranial window to a target area of a brain;

FIG. 2 is a block diagram showing at least a portion of the system of FIG. 1 providing feedback related to delivery of the light signal and/or the electrical signal;

FIG. 3 is an example perspective view of the window of FIG. 1 with an outer flange;

FIG. 4 shows example top view illustrations of differently shaped outer flanges of FIG. 3;

FIG. 5 shows example top view illustrations of magnetic alignment mechanisms incorporated into the differently shaped outer flanges of FIG. 4;

FIG. 6 shows an example magnetic alignment between the electro-optical applicator and the window for the transcutaneous delivery of the light signal and/or electrical signal through the electro-optical cranial window of FIG. 1;

FIG. 7 is a block diagram showing an example system with two electro-optical applicators and two electro-optical cranial windows each over a different hemisphere of the brain for delivery of a light signal and/or an electrical signal to two locations simultaneously and/or sequentially;

FIGS. 8 and 9 show examples of current flow through the system of FIG. 7;

FIG. 10 is a process flow diagram of a method for treating a neurological disorder with a system of FIG. 1;

FIG. 11 is a process flow diagram of a method for treating a neurological disorder with a system of FIG. 7; and

FIGS. 12 and 13 show illustrations of example uses of the systems of FIGS. 1 and/or 7 for delivering PBM and/or electrical stimulation to the precuneus.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an,” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “photobiomodulation”, abbreviated as “PBM”, can refer to the delivery of light signal(s) at one or more prescribed wavelengths and dosing schemes to a predefined target area within a patient's brain to achieve a desired physiological response (e.g., to treat at least one physiological condition).

As used herein, the term “electrical stimulation” can refer to the application of one or more electrical signals (e.g., current(s)) with one or more predefined parameters and dosing schemes via one or more electrodes to a predefined target area within a patient's brain to achieve a desired physiological response (e.g., to treat at least one physiological condition).

As used herein, the term external “electro-optical applicator” can refer to a device and/or part of a system external to the body of a patient that can generate, and in some instances configure, one or more PBM light signals and/or electrical stimulations to be delivered by one or more electro-optical cranial windows to a target area of a patient's brain.

As used herein, the term “electro-optical cranial window”, referred to more generally as “window”, can refer to a device that can be implanted under skin and into and/or at least partially through obstructive material of a patient's skull (e.g., the bone, dura, etc.) to deliver PBM and/or electrical stimulation from an external electro-optical applicator to a target area within the patient's brain. The window can include a light transmission region and an electrical transmission region. The window can also include one or more hardware, sensor, and/or communication mechanisms, and the like. In some instances, the window can be associated with one or more unique IDs identifying the window, the target area, the patient, one or more elements of a prescription for the PBM and/or electrical stimulation, or the like.

As used herein, term “light transmission region” can refer to a portion of a window with a high optical transparency (e.g., including a light pipe or the like) to facilitate transmission of one or more light signals (of PBM) with no or minimal attenuation. The light transmission region can include one or more optically transparent materials and/or components (e.g., glass, air, vacuum, transparent polymers, or the like) to convey the light signal(s) through the window to the target area to enable/enhance an effect of the PBM on the target area). In some instances, the one or more optically transparent materials and/or components can be shaped and/or doped to have one or more desired optical properties. In some instances, the light transmission region can have a low index of refraction or other properties that can enhance the transmission of light therethrough.

As used herein, the term “electrical transmission region” can refer to a portion of a window that can transmit one or more electrical signals to a target area of a patient's brain, including, but not limited to a conductive pathway to an electrode and the electrode, both of which are electrically insulated from the rest of the window.

As used herein, the term “obstructive material” can refer to any material, organic or inorganic, that blocks or otherwise hinders light delivery (e.g., by attenuating, refracting, resisting, etc.) and/or delivery of an electrical signal (e.g., as an insulator). Bone is an example of an organic obstructive material. In some instances, dura is another example of an organic obstructive material. Examples of inorganic obstructive materials can include, but are not limited to plates, screws, or other surgically implanted inorganic materials that can block or otherwise hinder light and/or electrical signal delivery.

As used herein, the term “dosing scheme” can refer to a schedule of one or more doses of PBM (e.g., quantities of light of one or more wavelengths) and/or doses of electrical stimulation (e.g., current at one or more parameters) to be delivered to a target area of a patient per a unit of time to treat the patient. A dosing scheme can include whether doses of the PBM and/or the electrical stimulation are applied simultaneously and/or sequentially, or a mixture thereof, a time between doses of PBM and/or electrical stimulation, one or more times of day when the dose of PBM and/or electrical stimulation is to be given, a quantity of PBM and/or electrical stimulation to be delivered, a target intensity of the light signal(s) and/or current to reach the target area, a luminance of a light source of the PBM, a power associated with the delivery of the PBM and/or electrical stimulation, an amount of PBM and/or current in the dose to reach the target area, or the like.

As used herein, the term “subcutaneous” can refer to something being situated or applied beneath (under) a patient's skin. For instance, something located subcutaneously is located within the patient's body under the skin). For example, the window described herein is subcutaneous.

As used herein, the term “transcutaneous” can refer to something being delivered through/across a patient's skin without physically disrupting the skin barrier (e.g., light and/or electrical signals can be delivered from an external opto-electrical applicator transcutaneously to a subcutaneous window for delivery to an internal target area).

As used herein, the term “light pipe” can refer to a mechanism that can transmit light lengthwise. Non-limiting examples of a light pipe can include optical fibers, transparent plastic rods, and the like. In some instances, light pipes can be coupled to one or more waveguides. In other instances, one or more waveguides may be used to facilitate light transmission without the light pipe.

As used herein, the term “patient” can refer to any warm-blooded organism, including, but not limited to, a human being, a pig, a rat, a mouse, a dog, a cat, a goat, a sheep, a horse, a monkey, an ape, a rabbit, a cow, etc. The terms patient and subject can be used interchangeably herein.

As used herein, the term “physiological condition” can refer to a disorder, disease, or patient state with a neurological component and/or symptom that is at least partially treated, ameliorated, or has its progression slowed by the application of PBM and/or electrical stimulation to one or more target areas of a patient's brain. Non-limiting examples of physiological conditions can include Alzheimer's disease, autism, schizophrenia, major depressive disorder (MDD), chronic pain, post-traumatic stress disorder (PTSD), attention deficit hyperactivity disorder (ADHD), and the like, when the target area is a part of the default mode network (DMN). Other non-liming examples can include the target area being cortical areas affected by stroke, such as the motor cortex, the prefrontal cortex to treat depression or other psychiatric disorders, any cortical region affected by a traumatic brain injury (TBI), or the like.

As used herein, the term “target area” can refer to a portion of a patient's brain chosen to receive one or more doses of PBM and/or electrical stimulation according to the dosing scheme to treat a pathology. Target areas can be of differing sizes, depths, locations, and/or cell compositions depending on the physiological condition to be treated. As an example, one or more target areas may be in a cortical area of the brain. For instance, the one or more target areas can be within a default mode network (DMN), such as in the precuneus region of the brain. For instance, target areas can be in a same or different region of both hemispheres of the brain that can be reached employing one or more windows. Other targets areas can include cortical areas affected by stroke, such as the motor cortex, the prefrontal cortex to treat depression or other psychiatric disorders, any cortical region affected by a traumatic brain injury (TBI), or the like.

### II. Overview

Photobiomodulation (PBM) and/or electrical stimulation provide an attractive solution for treating pathologies of various target areas (e.g., tissues within a patient's body). For example, disruptions of the Default Mode Network (DMN) in the brain are thought to be related to numerous pathologies, including Alzheimer's disease, autism, schizophrenia, major depressive disorder (MDD), chronic pain, post-traumatic stress disorder (PTSD), attention deficit hyperactivity disorder (ADHD), and the like. PBM and/or electrical stimulation are each treatment modalities that could modulate the DMN to at least partially treat such disruption. However, both PBM and electrical stimulation face significant challenges in application to certain areas of the brain, with current solutions being fully implanted and very invasive or fully external and entirely non-invasive. Fully external and fully implanted applications of PBM and/or electrical stimulation experience many difficulties particularly with respect to targeting the brain. Implanted light and/or electrical sources underneath obstructive material(s) have inherent complexities (e.g., costly, requires surgery, limited lifespan, poor power use, etc.) that make implanted light and/or electrical sources less preferable to external sources that have fewer complexities. However, traditional external light and/or electrical sources have a limited range and efficacy, including an inability to properly penetrate through obstructive materials of the body (e.g., the skull, dura, etc.). Moreover, it is nearly impossible to accurately estimate the amount of light and/or electrical signal lost to the obstructive material and/or the amount of light and/or electrical signal actually delivered to the target area by a fully external system.

In contrast, PBM and/or electrical stimulation can be delivered to a target area of a brain through obstructive material of a patient's skull (bone, dura, etc.) more safely and effectively using a system with one or more external electro-optical applicators and one or more electro-optical cranial windows (also referred to as windows). The one or more external electro-optical applicators can generate and transfer to the one or more windows the one or more of PBM and/or electrical stimulation. The one or more windows can be positioned through the obstructive material between skin of the patient and the target area and can be configured to deliver the one or more of PBM and/or electrical stimulation to the target area. The one or more windows can deliver a PBM light signal from an external light source to the target area in a safer manner and with significantly less attenuation due to the obstructive material than traditional PBM application. The one or more windows can alternatively and/or additionally transmit an electrical signal to the target area using an electrode located in/on the window and not implanted into brain tissue for less invasive electrical stimulation.

### III. Systems

One or more electro-optical cranial windows (also referred to as “windows”) through a skull of a patient can be used deliver one or more of photobiomodulation (PBM) and electrical stimulation to a target area within a brain of a patient to treat one or more neurological disorders. PBM generally refers to the delivery of light, at prescribed wavelengths and dosing schemes (e.g., amounts per time), to the target area to achieve a desired physiological response. Electrical stimulation generally refers to the delivery of an electrical signal (e.g., current) having one or more parameters with prescribed dosing schemes to the target area to achieve a desired physiological response. Photobiomodulation and electrical stimulation can, in some instance, be combined as a therapy and/or treatment for various neurological disorders when applied to one or more target areas of the brain. For instance, target areas of the brain can include, but are not limited to, one or more portions of the Default Mode Network (DMN) when the neurological disorder is one of Alzheimer's disease, autism, schizophrenia, major depressive disorder (MDD), chronic pain, post-traumatic stress disorder (PTSD), attention deficit hyperactivity disorder (ADHD), and the like. The DMN can include the dorsal medial prefrontal cortex, posterior cingulate cortex, precuneus, and angular gyrus. For instance, the target area can be the precuneus to treat dementia. Other targets areas can include cortical areas affected by stroke, such as the motor cortex, the prefrontal cortex to treat depression or other psychiatric disorders, any cortical region affected by a traumatic brain injury (TBI), or the like. It should be understood that PBM and/or electrical stimulation can be delivered to one or more target areas at a same or different time and at each of the different target areas at the same or different wavelengths, parameters, and dosing schemes as treatment for the same or different pathologies using the same or different electro-optical applicator(s) and/or window(s).

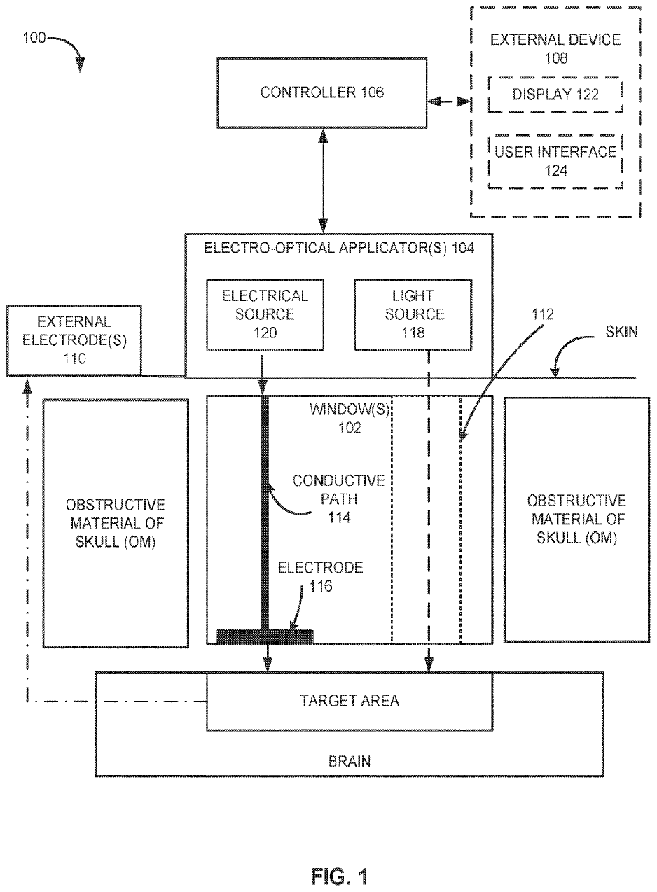

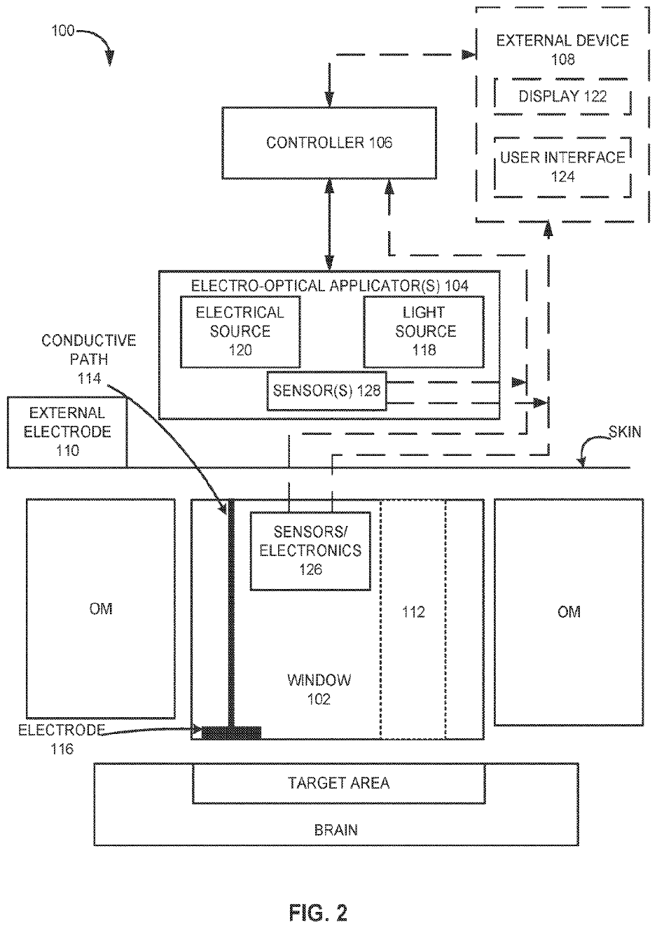

FIG. 1 shows an example system 100 for delivering PBM and/or electrical stimulation to a target area of a patient's brain using a partially external and partially subcutaneous system for improved efficacy. The system 100 includes at least one subcutaneously implanted electro-optical cranial window 102 and at least one external electro-optical applicator 104. With reference to FIG. 1 (as well as FIGS. 2-6), a single window 102, electro-optical applicator 104, target area, and other components are described, but it should be understood that any number one or greater can be used/envisioned unless otherwise specifically stated. When two or greater window(s) 103 are utilized, the windows can be positioned over the same and/or different target areas of the same or different hemispheres of the brain. The electro-optical applicator 104 can be in communication, wired and/or wireless, with a controller 106 (as shown) or can be embodied in a single device with the controller. In some instances, the controller 106 can additionally be in wired and/or wireless communication with an external device 108 (e.g., a mobile device, such as a smart phone, a tablet, a laptop, or the like, associated with a patient, a medical professional, or the like, that at least can provide input to the controller 106). In other instances, the controller 106 can be and/or can include a power source and communicatively connect the electro-optical applicator 104 with the external device 108, letting the external device act as the “controller”. At least one external electrode 110 can be positioned on the skin of the patient as a common and/or return electrode for the electrical stimulation.

The electro-optical applicator 104 can be external to the patient and, for instance, can be positioned on, adjacent, or near the skin of the patient and aligned with the window 102 underneath the skin. The electro-optical applicator 104 can include at least one electrical source 120 (e.g., an electrical signal generator) and at least one light source 118 (e.g., at least one LED or the like capable of generating one or more wavelengths of light) for generating the electrical stimulation and the light signal of the PBM, respectively. While the electro-optical applicator 104 is illustrated as contacting the external side of the skin, it should be understood that the electro-optical applicator may be held a distance above the skin or, in some instances, a portion of the electro-optical applicator (such as the light source 118 and/or the electrical source 120, may be minimally invasively implanted under the skin but still external to the skull.

The window 102 can be subcutaneously implanted (e.g., beneath the skin of the patient) in the skull of the patient to span at least part or all of the space from at least beneath the skin through the obstructive material of the skull. It should be noted that although the skin does scatter light and is the primary absorber of light, especially darker skin, for these purposes, the skin is not considered a significant obstruction for light and/or certain electrical signals to pass through. Light can attenuate rapidly with distance through tissue (e.g., the more tissue the greater the attenuation) (and attenuation can be based on the type of tissue as well), but a significant portion of the non-target intervening tissue, such as bone, dura, and the like (also metal plates as an inorganic example), can be removed, allowing for better light transmission. Additionally, the electrical conductivity of bone is relatively low, so the window 102 improves transmission of the electrical signal to the target so that the target receives therapeutic electrical signal doses. The window 102 can span through the obstructive material and can create a path for light and/or electrical transmission from outside the patient's skull to the target area of the patient's brain. The window 102 can include at least a light transmission region 112 and an electrical transmission region positioned and configured to not interfere with each other. The light transmission region 112 can be a high optical transparency region and can include at least one optically transparent region, material, and/or component for transmitting the light signal of the PBM from the at least one light source 118 through the window 102 to the target area of the brain of the patient.

The electrical transmission region can include at least one conductive path 114 from a conductive access at the “top” (e.g., side closest to the skin) of the window 102 to the at least one electrode 116 and the at least one electrode. The at least one electrode 116 can be positioned proximal to the target area of the brain of the patient. For instance, at least a portion of the at least one electrode 116 can be positioned at and/or near a bottom of the window 102 near and/or adjacent the target area of the patient's brain. Within the window 102 the electrical transmission region can be electrically insulated around the conductive path 114 and the electrode 116 so that the lowest impedance path for the current is to go in the top of the window, through the electrode of that window to the target area of the brain, through the target area of the brain (causing the stimulation effect), and out through the return, external electrode 110. Not shown, the current can then, in some instances, be returned to the electro-optical applicator 104 (e.g., from the external electrode 110). The electrical transmission region can deliver an electrical modulation from the at least one electrical source 120 via the at least one electrode 116 to the target area of the patient's brain. The at least one electrode 106 can be biocompatible and configured to not cause harm to the brain or surrounding tissues at therapeutic current levels.

A light signal and/or an electrical stimulation can be configured (according to a prescription that can be, for example, input into the controller 106) and delivered to the target area of the brain to treat dementia, Alzheimer's disease, autism, schizophrenia, major depressive disorder, chronic pain, post-traumatic stress disorder, or the like. As noted, the light signal and/or the electrical signal can be externally delivered to the window 102 by at least the electro-optical applicator 104 in at least communication with the controller 106. The electro-optical applicator 104 and the controller 106 can be embodied as a single device or as separate but connected (according to wired and/or wireless communication) devices. The controller 106 can include a non-transitory memory and/or processor (not shown) that can be configured to set at least one light signal parameter and/or at least one electrical modulation parameter and control application of the light signal and/or the electrical modulation. The at least one electrical modulation parameter can include at least one of a polarity, an amplitude, a pulse width, a pulse timing, a pulse rate, a pulse pattern, and/or selection of one or more of the at least one electrode 116. The at least one light signal parameter comprises at least one of a power, a duration, a pulsatile delivery scheme, a temporal delivery scheme, a wavelength, and/or a timing of light signal delivery. The controller 106 can, for instance, regulate a timing of the electrical signal delivery in concert with a timing of the light signal delivery. Delivery of the electrical stimulation can be concurrent with transmission of the light signal and/or separate from transmission of the light signal. The controller 106 can include a battery and/or can connect to a power source (e.g., line power, an external battery, or the like) and can power the electro-optical applicator 104. In some instances, the controller 106 can perform an impedance and/or a continuity check of at least a part of system 100 and may not allowed generation of a light and/or electrical signal until impedance and/or continuity are within predefined appropriate levels. In some instances, the electro-optical applicator 104 can send feedback data (e.g., information about the state of the electro-optical applicator, information about the patient, information about the window, the unique ID of the window, etc.) to the controller 106.

The light source 118 (e.g., one or more LEDs, etc.) of the electro-optical applicator 104 can be configured to deliver the light signal to the target area via the window 102 transcutaneously through the patient's skin. In some instances, the electro-optical applicator 104 and/or the controller 106 can include one or more indicators (either physical indicators (e.g., visual, auditory, and/or tactile) and/or indicators/flags that are shown on a display), such as an ON/OFF indicator, a charge status indicator, a fault status indicator (e.g., indicating the one or more components of the system is not working), a dosing scheme indicator (e.g., indicating a dose is needed, where the patient is in the dosing scheme, etc.), or the like. In some instances, the controller can include a user interface and/or display for inputting and displaying such information. In other instances, the controller 106 can be in wired and/or wireless communication with an external device 108, such as a personal computer, smartphone, tablet, or the like, that can be used to input information (such as parameters, dosing schemes, etc.), view outputted information (such as sensor readings, actual dosing information, etc.), and/or further analyze outputted information. The external device 108 can include a non-transitory memory and processor (not shown), a display 122, a user interface 124, a haptic feedback device for tactile alerts (not shown), and/or a speaker device for audio alerts/information (not shown). The external device 108 can be used by a medical professional to input and/or change aspects of the therapy. In other instances, the external device 108 can belong to a patient and can track information related to the therapy (dosing information, applicator power, etc.). In some instances, the external device 108 can be used by the patient to change aspects of the therapy within bounds set by the medical professional.

As briefly discussed above, the skull and at least some surrounding tissues include obstructive materials that lie between the skin and target areas of the brain. Obstructive materials can stop at least a portion of a light signal (and in some instances an electrical signal if the obstructive materials are insulating) from passing through to the target area by attenuation, refraction, resistance or the like. Examples of the obstructive material can include tissues such as bone of the skull, muscle, fat, or the like and/or foreign objects such as metal plates or other surgically implanted materials. In the main cases described herein the skin is thin (~1-4 mm thickness) and it is desirable to leave intact because it performs an important function as a barrier to infection. For reasons described above, skin is not considered a significant obstruction for light/electricity. However, it should be noted that in some instances the skin may provide a small level of obstruction to certain light signals and/or electrical signals, which can be detrimental to dosing schemes of PBM that deliver smaller amounts of light and/or electrical signals (e.g., the level of the obstruction of the skin may be on the scale of or significantly impact the delivery of the smaller dosage) (in these situations, the light source 118 and/or the electrical source 120 of the electro-optical applicator 104 can be minimally invasively implanted under the skin). It should be noted that the absorption coefficient for skin may not always be dramatically different than other tissues, but the thickness of skin is usually thinner than what is traditionally thought of as obstructive material (e.g., bone). For instance, skin on the scalp may be on the order of 1-1.5 mm thick, while bones of the skull can be quite thick comparatively, on the order of 3-11.5 mm.

As noted, the general case illustrated in FIG. 1 (as well as FIGS. 2, and 7-9) shows the obstructive material positioned between the skin and the target area. Removing a portion of the obstructive material removes an obstacle for the light and electrical signals to reach the target area of the patient's brain (e.g., an obstacle that can hinder, attenuate, refract, resist, or the like, an amount of the light and/or electrical signal before it reaches the intended target area). As shown in FIG. 1 (as well as FIG. 2), the window 102 can be placed within and through at least a portion of the obstructive material in a space that spans through the obstructive material at a predetermined location (e.g., in line with at least a portion of the target area, in line with one or more physiological landmarks, on a line between at least a portion of the light source 118 of the electro-optical applicator 104 and the at least a portion of the target area, or the like). At the predetermined location, a hole through at least a part of the obstructive material can be pre-existing (e.g., based on a prior accident or a prior surgical intervention) or can be created in the obstructive material (e.g., surgically) and the window 102 can be placed within the hole.

The light transmission region 112 of window 102 can at least partially include at least one of gas, liquid, glass, crystal, a non-material empty space like a vacuum, or any sufficiently light transmissive material and/or light pipe for light to pass through to the target area without significant obstruction that would change the efficacy of the light. By spanning through the hole, the light transmission region 112 can allow a greater amount of light to pass from the light source 118 of the electro-optical applicator 104 to at least a portion of the target area (as compared to if the light signal had to pass through the obstructive material). By replacing a portion of the obstructive material, the substantially clear material of the light transmission region 112 of window 102 can remove an obstacle for the light signal and allow a greater amount of the light signal to pass to the target area The electrical transmission region can transmit the electrical signal from electrical source 120 thought the conductive path 114 and the electrode 116 to the target area. By removing less conductive materials and providing a designated conductive path the electrical signal can more effectively reach the target area of the brain through the electrical transmission region compared to surface electrodes alone. Additionally, by the electrode 116 not being implanted into the brain itself, any of the potential side effects of brain implantation can be avoided. It should be understood that at least the conductive path can be insulated. However, in some instances, the conductive path and the light transmission region 112 can each be insulated/shielded (e.g., the outer window 102 can be made of an insulating material and may include additional insulating material around the conductive path 114 and/or the light transmission region 112).

In other instances, the light transmission region 112 of the window 102 can also include at least a portion of at least one optical feature (also referred to as a lens component) to reflect, focus, and/or spread the light signal before delivery to the target area of the patient. The optical feature can include a mirror, a lens, a diffusor, or the like, arranged based on the ultimate function desired. As an example, the optical feature can be a flat lens, such as a GRIN lens, or a non-flat lens, such as a Fresnel lens, which can focus the light. The flat lens can focus the light at a specific angle and/or at a specific part of the target area or to a light pipe and/or waveguide (now shown) connected to or in communication with the window 102 to deliver the light signal to the target area. The non-flat lens, such as the Fresnel lens, has a possible advantage of using simpler materials even though the non-flat lens has a non-flat surface. The window 102 may, additionally or alternatively, include a metamaterial (one or more of a class of artificial materials that can achieve electromagnetic properties that do not occur naturally, such as negative index of refraction or electromagnetic cloaking) to focus or spread the light and can be selected based on tissue properties within the target area and whether focusing or spreading is preferred. The metamaterial, in some instances, can be chosen based on tissue properties discovered based on preoperative imaging of the target area.

The window 102 can extend through the obstructive material without contacting and/or penetrating the target area. Although the window 102, the obstructive material, the skin, and the target area are illustrated as being separated from each other by at least one distance; however, this is simply for ease of illustration. It should be understood that the distances can each be any number from zero (e.g., touching/contiguous with at least one of each other) to a gap of about 200 mm, or more, depending on one or more materials of the window 102, dimensions of the window, tissue regrowth, positioning of the window, location of the target area, etc. In some instances, the target area may be a further distance from the obstructive material and the window 102 and a light pipe and/or waveguide, as discussed above, may be part of the system extending from the light transmission region 112 for transmitting the light signal to the target area.

The window 102 can be a permanent implant or the window can be a temporary and removeable implant (e.g., the obstructive material may be allowed to heal—partially or completely—after removal of the window to fill the hole or the hole may exist forever). For example, at least a portion of the window 102 can include a bioresorbable material that starts to bioresorb after a time duration. For example, the window 206 can be bioresorbable to enable conveyance of an amount of light and/or the electrical signal for a finite, predefined period of time (e.g., based on one or more properties of the bioresorbable material, where the bioresorbable material can be chosen based on the therapeutic application). In some instances, the window 102 can additionally hold one or more drugs (e.g., in one or more reservoirs, in a substrate matrix, or the like) and can deliver the one or more drugs to the target area or tissue near the target area before, concurrent with, and/or after delivery of the light signal and/or electrical signal to the target area for additional therapeutic effect. In other instances, the controller 102 can signal another device (not shown) to deliver a drug to the patient (e.g., to a location not in or near the target area) before, concurrent with, and/or after delivery of the light signal to the target area.

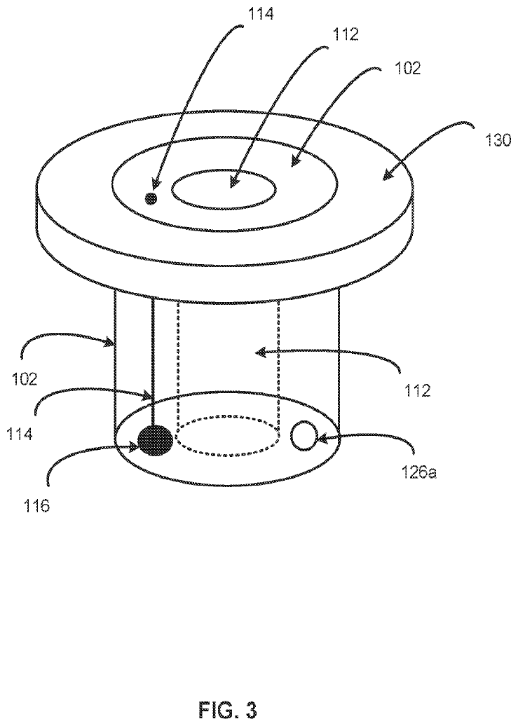

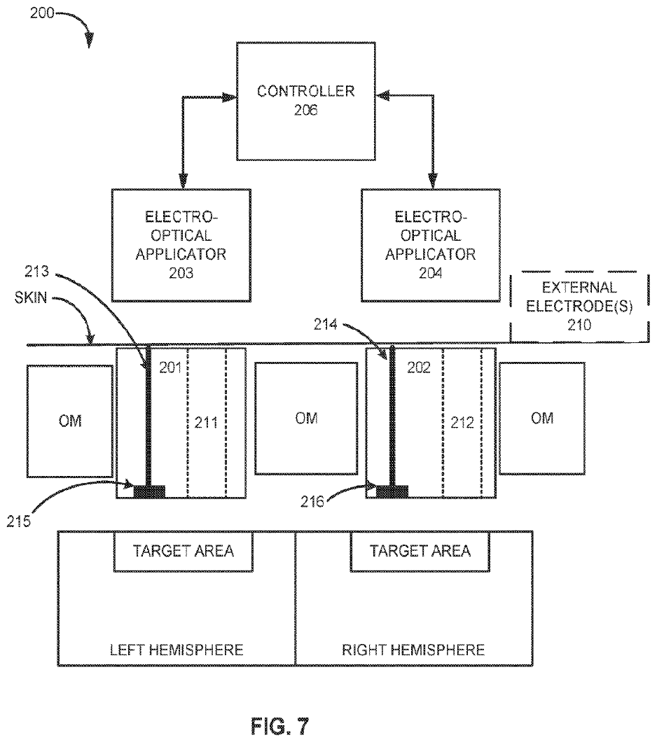

It should be understood that while the side view of the window 102 (and windows 201 and 202 in FIG. 7) are shown with a rectangular shape, any shape that can span through the obstructive material can be used. In some instances (e.g., to simplify the implant procedure), the window 102 can have a generally round cross section and a cylindrical length (e.g., as shown in FIG. 3). When the window 102 has a generally round cross section, the implant procedure can include drilling a hole in the obstructive material to create a space and placing the window 102 in the space. When the cross section of the window 102 is a round cylinder, the window can fill at least a portion of the hole drilled through the obstructive material. For example, a cross section of the window 102 can have a diameter less than or equal to a diameter of the hole. The drill can have a diameter the same as, or about the same as, a typical burr hole drill (and thus require no new equipment for a surgeon to place). For instance, the diameters for the hole and window 102 can be, but are not limited to, 9 mm, 11 mm, 14 mm, 16 mm, 22 mm, 25 mm, and the like. As an example, the inside boundary of the light transmission region 112 of window 102 can be reflective so that light that is inside the window can continue through the window instead of being absorbed by the window when light hits the internal boundary.

In some instances, the window 102 can include only passive electronic components (e.g., shown in FIG. 1). In other instances, the window 102 can include active electronic components such as sensors/electronics 126, (e.g., one or more electronic components configured to receive external power) (as shown generally as sensors/electronics 126 in FIG. 2). As shown in FIG. 2, the sensors/electronics 126 can include one or more sensors in the window 102 and accompanying circuitry for running the one or more sensors and communicating recorded data back to the controller 106 or in an external device 108 as feedback. The sensor(s) 128 can include one or more sensors in the electro-optical applicator 104, as well as accompanying circuitry for running the one or more sensors and communicating recorded data back to the controller 106 or external device 108 for feedback. The one or more sensors can include, for instance, a temperature sensor, a photodetector, a reflector, a current sensor, or the like. For example, the feedback sent from the window 102 and received by the controller 106 can include information (data signal or other type of signal) about light received by the window, transmitted through the window, exiting the window, or the like. The feedback can allow the controller 106 to configure and/or reconfigure the light and/or electrical signal parameters more precisely so that the light signal and the electrical signal delivered to the target area matches the prescription. As an example, at least a portion of the feedback can be embodied as light reflected back to the controller 106 and/or the electro-optical applicator 104 (wherein the controller and/or the electro-optical applicator can further include a photodetector (not shown) to detect at least one property of the reflected light). In another example, a temperature sensor can be configured to sense a temperature of the window 102 and/or the target area, and the controller 106 can be configured to determine the at least one light signal and/or at least one electrical signal parameter based on a pre-determined temperature management limit for the target area and/or the window itself. In a further example sensor(s) 128 can include a temperature sensor that can be configured to sense a temperature of the skin between the electro-optical applicator 104 and the window 102 and the controller 106 can determine if at least one light signal and/or at least one electrical signal parameter needs to be changed, and make said change, based on a pre-determined temperature management limit for the skin (e.g., before a threshold level of damage occurs).

The sensors/electronics 126 can send feedback to the controller 106 and/or the external device 108. In one instance the feedback can include an indication of the light transmitted to the target area and/or the electrical signal transmitted to the target area. The indication of the light transmitted to the target area may be determined by direct measurement (e.g., if a light sensor component is positioned near and/or in at least a portion of the target area) or indirect measurement (e.g., if the light sensor component is positioned in/on at least a portion of the window 102) by taking the total light delivered from the electro-optical applicator 104 and subtracting at least the amount of the light reflected or absorbed by the light sensor component. The indication of the electrical signal transmitted to the target area may be determined by direct or indirect measurement with one or more additional electrodes within the window 102, an external electrode 110, or an additional implanted electrode (e.g., near the target area of the brain). Any circuitry related to/within the one or more light and/or electrical sensing components of sensors/electronics 126 can relate the reflection or absorption of light or the current flowing therethrough, respectively, to the controller 106 and/or the external device 108 by wireless transmission. For instance, the light and current throughput through the window 102 to the target area can be estimated by the controller 106 and/or external device 108 based on the feedback and then, if determined to be necessary by the controller and/or external device one or more light and/or electrical signal parameters can be altered.

The feedback, or other data, from the window 102 and/or the sensors/electronics 126 and/or sensor(s) 128 can, additionally or alternatively, include, but is not limited to, information about the window (e.g., dimensions, materials, etc.), information about the patient (e.g., age, gender, condition, medical notes, prescription information, etc.), information indicating an alignment of the light source of the electro-optical applicator 104 and the window (e.g., direct alignment, partial alignment, etc.), or the like. For example, at least a portion of data can be submitted to the controller 106 before the first transmission of the light and electrical signals from the electro-optical applicator 104. The controller 106 can monitor the amount, intensity, direction, duration, time of application, etc., of the light signal and/or the amount, amplitude, duration, frequency, pulse timing, etc. of the light signal based on a feedback signal(s) and can adjust the configuration of at least one light signal parameter (e.g., wavelength, intensity, time of application, duration of application, pulsed/solid, etc.) and/or electrical signal generator (e.g., a polarity, an amplitude, a pulse width, a pulse timing, a pulse rate, a pulse pattern, selection of one or more of the at least one electrode, etc.) being generated by the electro-optical applicator 104 according to the feedback signal received. As such, the controller 106 and the window 102, including sensor(s)/electronics 126 and/or the electro-optical applicator 104 including sensor(s) 128 can be a closed loop system that can ensure that a certain PBM and/or electrical dosage profile has been delivered to the target area.

However, it should be understood that the controller 106, in other instances, can be an open loop system (e.g., user in the loop) where at least a portion of the data is presented to a user (patient and/or medical professional) (e.g., via external device 108, who makes adjustments to the controller 106 based on the data. The controller 106, in some instances, can be programmed with a prescription for the PBM dosage profile (e.g., light intensities wavelengths, times of application, lengths of light application, types of light application, etc.) and/or the electrical modulation dosage profile (e.g., a polarity, an amplitude, a pulse width, a pulse timing, a pulse rate, a pulse pattern, selection of one or more of the at least one electrode, etc.). In some instances, the prescription can include a finite number of doses and a prescription to deliver the finite number of doses at specified times, after which the patient must see a clinician, talk to a clinician, etc., to receive a refill, a new prescription, or the like. The feedback can be used to affect the dosing signal, for example, if it is determined not enough light or current is reaching the target area at a time then the light or current can be made more intense, applied for a longer time, etc. or if the wrong wavelengths are detected then the wavelength of the light being applied can change, or if the light signal being applied is too intense then the controller can decrease the intensity.

In another instance, the controller 106 can store one or more such PBM dosage profiles. For example, the controller 106 can store different PBM dosage profiles for different target areas and/or patients. As an example, a patient may have two different target areas requiring treatment with two different windows (e.g., windows 201 and 202 discussed in more detail below) implanted through obstructive material above each target area, and each correspond to a different PBM dosage profile. The initial feedback can include a unique ID of the specific window. The controller can identify the specific window based on the unique ID and match the specific window to the correct PBM dosage profile, ensuring that the correct target area receives the correct PBM dosage.

As previously mentioned, the window 102 can also include an outer flange 130, as shown in FIGS. 3-5. The outer flange 130 can at least one of secure the window 102 in place at the implanted location (e.g., a hole through the obstructive material) and improve a fit of the window in the implanted location (e.g., a hole through the obstructive material). The outer flange 130 can, additionally or alternatively, include features for securing the assembly to bone (e.g., holes so that screws, like titanium screws, can secure the window to a portion of the skull or other bone) or other obstructive material. The outer flange 130 can be, for instance, a flat rim, collar, or rib extending from a portion of the top of window 102. The outer flange 130 can also include one or more alignment mechanisms to facilitate correct alignment of the electro-optical applicator with the window 102. For instance, as shown in the three-dimensional example of the window 102 shown in FIG. 3, the window 102 can be a cylinder and the outer flange 130 can extend from a portion of the sides of the window near the top of the window. It should be understood that FIG. 3 is only an example and any shapes and/or dimensions that would fit for a given hole through the obstructive material are anticipated. The body of the window 102 includes the light transmission region 112 and the electrical transmission region as described above (it should be understood that the body is see-through for simplicity of drawing and explanation and may not be see through in practice).

In FIG. 3 the light transmission region 112 is positioned through a center of the window 102 and the electrical transmission region is offset to one side of the window, as shown by the conductive path 114 and the electrode 116. However, the light transmission region 112 and the electrical transmission region can be in any configuration where the two regions do not interfere with one another transmitting their respective signals. The conductive path 114 is shown exposed at the top of the window 102, but this is only one example. For instance, the conductive path 114 may be fully encapsulated by the window and an access device for picking up the electrical signal from the electro-optical applicator 104 may be included. The electrode 116 is shown as a point electrode extending through a portion of the bottom of the window 102 to deliver the electrical signal to the target area, but it should be understood that any electrode shape and/or configuration, including multiple electrodes, for delivering the electrical signal to the target area is considered. A temperature sensor 126a is shown in FIG. 3 on a portion of the bottom of the window 102. It should be understood that other sensors, not shown, can be positioned at other locations within and/or on the window 102. For instance, the temperature sensor 126a can sense a temperature of the target area and wirelessly send the sensed temperatures back to the controller (not shown in FIG. 3) where the controller can determine the at least one light signal parameter and/or the electrical signal parameter based on a pre-determined temperature management limit for the target area.

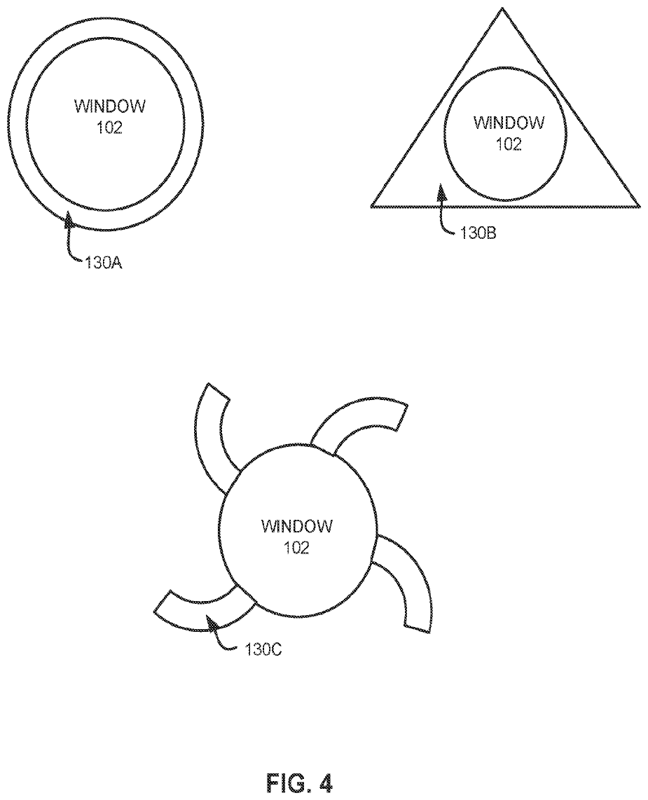

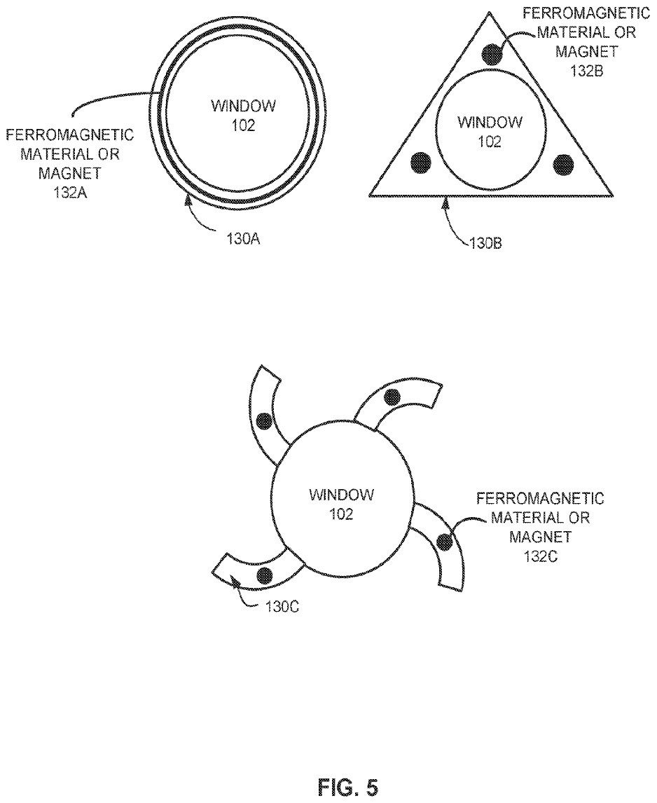

FIGS. 4 and 5 show different example layouts of top views of the outer flange 130 (130A, 130B, and 130C) with respect to the window 120. In the top left example, the outer flange 130A is a ring concentric around the perimeter of the window 102. In the top right example, the outer flange 130B can have a triangular top view surrounding at least the perimeter of the top of the window 102 (shown here as circular). In the bottom example, the outer flange 130C can include a plurality of protruding arms. Four curved protruding arms are shown, but any number and/or shape that can secure the window to the skull can be imagined. FIG. 5 shows each of the outer flange examples 130A, 130B, and 130C including one or more ferromagnetic materials and/or magnets 132A, 132B, and 132C, respectively, as example alignment mechanisms. It should be understood that the example layouts in FIGS. 4 and 5 are non-exhaustive and not intended to be limiting.

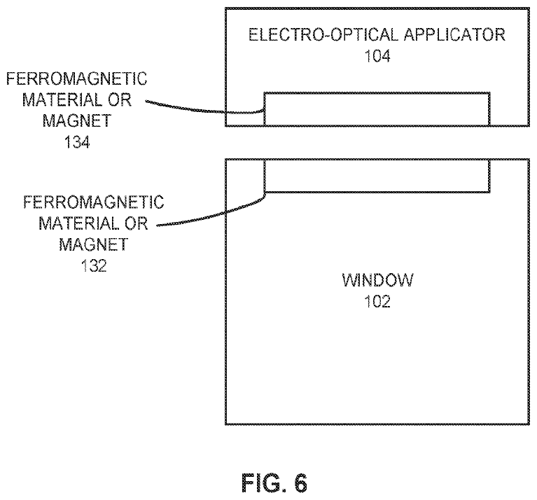

As shown in the block diagram of FIG. 6, the electro-optical applicator 104 can be positioned above the window 102 and can be configured to align with the window 102. The alignment can be such that at least one light source (e.g., light source 118) of the electro-optical applicator 104 is aligned with at least the light transmission region (e.g., 112) of the window 102 and/or the electrical source (e.g., electrical source 120) is aligned to deliver the electrical signal to the conductive path of the window (e.g., conductive path 114). The electro-optical applicator 104 can be aligned with the window 102 mechanically, magnetically, or the like. The alignment can be done, for instance by the patient, a medical professional, and/or a caregiver, by gently pressing the electro-optical applicator 104 onto the window 102 (under the skin). The mechanical alignment can be based on a mechanical feature or mechanism within the window 102 and/or the electro-optical applicator 104. The magnetic alignment can be based on at least one ferromagnetic material on one side of the skin and a magnet on the other (opposite) side of the skin. For example, at least a portion of the window 102 can include a ferromagnetic material and at least a portion of the electro-optical applicator 104 can include a magnet. As another example, at least a portion of the electro-optical applicator 104 can include a ferromagnetic material and at least a portion of the window 102 can include a magnet. As a further example the electro-optical applicator 104 and the window 102 can both include one or more magnets, where the North and South poles in each are arranged to facilitate alignment (not repulsion). The mechanical and/or magnetic features and/or mechanisms can be non-corrosive and/or non-erodible (or resistant to corrosion and/or erosion) and/or may have rounded (not-sharp) edges for maximum comfort and minimal tissue damage. As shown in FIG. 5, if the window 102 also includes an outer flange 130A, 130B, 130C, then the ferromagnetic material or the magnet 132A, 132B, 132C of the window 102 (depending on if the electro-optical applicator includes a magnet or ferromagnetic material, respectively) can be positioned in at least a portion of the outer flange. For instance, a ferromagnetic or magnetic ring 132A is shown in/on outer flange 130A, multiple discrete instances of ferromagnetic material or magnets 132B are shown in/on outer flange 130B, and one instance of ferromagnetic material or magnet 132C are shown in/on each arm of outer flange 132C. It should be understood that the example layouts in FIG. 5 are non-exhaustive and not intended to be limiting.

Referring now to FIG. 7, an example system 200 is shown that includes two windows, 201 and 202, that can be positioned to deliver light (PBM) and/or electrical signals to target areas in different hemispheres of the brain (e.g., left and right) for therapeutic effect. For instances the target areas can be the precuneus of the right and the left hemispheres of the brain, for additional and/or alternative PBM and/or electrical modulation effects from use of a single window (as shown in FIGS. 1 and 2). In system 200, each of the windows 201 and 202 can be aligned with a separate electro-optical applicator 203 and 204, each comprising at least one light source and electrical source (as shown with respect to electro-optical applicator 104 in FIGS. 1 and 2). It should be understood that a single electro-optical applicator with multiple light source and electrical sources could be used instead of the target areas are close enough together. Each of the electro-optical applicators 203 and 204 can be in communication with a controller 206 (in some instance, not shown, each electro-optical applicator can be in communication with its own controller and/or a designated portion of the controller 206). Each of the windows 201 and 202, electro-optical applicators 203 and 204, and controller 206 can include any and/or all of the components, aspects, and/or configurations described previously with respect to FIGS. 1-6.

The first window 201 can span partially and/or fully through obstructive material of the skull of the patient (e.g., bone, dura, etc.) between the skin and the target area of a hemisphere of the brain (e.g., a portion of the precuneus of the left hemisphere of the brain). The second window 202 can span partially and/or fully through obstructive material of the skull of the patient (e.g., bone, dura, etc.) at a different position in the skull, between the skin and another target area of a hemisphere of the brain (e.g., a portion of the precuneus of the right hemisphere of the brain). It should be understood that each of the first and second windows 201 and 202 may have a top that extends near and/or in contact with the skin or in line with the top of the obstructive material, and a bottom that extends near and/or in contact with the target area or in line with the bottom of the obstructive material, depending on clinical usage. Each window 201 and 202 can include a light transmission region 211, 212 and an electrical transmission region to transmit a light signal for PBM and/or an electrical signal, respectively. The first window 201 can include first light transmission region 211 and first electrical transmission region, that can include a first electrode 215 positioned proximal to the target area of the hemisphere to deliver the electrical stimulation and a conductive pathway 213 from the top of the first window to the first electrode. The second window 202 can include a second light transmission region 212 and a second electrical transmission region, that can include a second electrode 216 position proximal to the target area of the other hemisphere to deliver the electrical stimulation and a conductive pathway 214 from the top of the second window to the second electrode. It should be understood that each of the first and second windows 201 and 202 can include one or more electrodes, but only one is shown and described for simplicity. Additionally, system 200 can include one or more external electrode (external electrode(s)) 210 positioned on the skin of the patient, for example near at least one of the target areas.

The first electro-optical applicator 203 can include a light source (not shown) and an electrical source (not shown), as described in detail above with respect to FIGS. 1 and 2, and can align the light source with the first window 201 to deliver a light signal through the light transmission region 211 of the first window to the target area of the left hemisphere and the electrical source with the conductive pathway 213 to the target area of the left hemisphere. The second electro-optical applicator 204 can include another light source (not shown) and another electrical source (not shown), as described in detail above with respect to FIGS. 1 and 2, and can align the other light source with the second window 203 to deliver a light signal through the light transmission region 212 of the second window to the target area of the right hemisphere and the electrical source with the conductive pathway 214 to the target area of the right hemisphere. The first electro-optical applicator 203 and the second electro-optical applicator 204 can both, or separately, be configured to be positioned on, near, or pressing into the skin above their respective windows 201 and 202. In another instance a portion of the electro-optical applicators 203 and 204 may be positioned under the skin, such as the light source, and in communication with other components of the electro-optical applicators.

The controller 206 can be in wired and/or wireless communication with the first electro-optical applicator 203 and the second electro-optical applicator 204. The controller 206 can, for instance, set at least one light signal parameter for the first light source of the first electro-optical applicator 203 and another at least one light signal parameter for the second light source of the second electro-optical applicator 204 and at least one electrical stimulation parameter for the first electrode of the first electro-optical applicator and/or another at least one electrical stimulation parameter for the second electrode of the second electro-optical applicator. The light and electrical signal parameters can be the same and/or different for each electro optical applicator, 203 and 204. The light signal parameters and the electrical signal parameters can be determined based on a manual input (e.g., through a user interface of the controller 206 or associated with controller 2016) or based on one or more closed loop inputs (e.g., from one or more sensors of the windows 201 and/or 202 (not shown), separate physiological sensor(s), a predetermined timed prescription dosing scheme, or the like).

The system 200 can be used in several different configurations for therapeutic effect when applied as shown to two target areas (on different hemispheres as shown, or on the same hemisphere (not shown)). In other instances, not shown, the system 200 can include more than two windows and each of the windows can be positioned on a same and/or different hemisphere of the brain and over a same and/or different target area (e.g., locations), depending on the use and the physiological condition(s) to be treated. The PBM light can be applied by the first and/or second electro-optical applicators 203 and 204 through one or both of the windows 201 and/or 202 simultaneously and/or separately. The first and/or second electro-optical applicators 203 and 204 can apply the first and/or the second electrical signals via the electrodes of the windows 201 and 202 simultaneously, separately, and/or from one or the other.

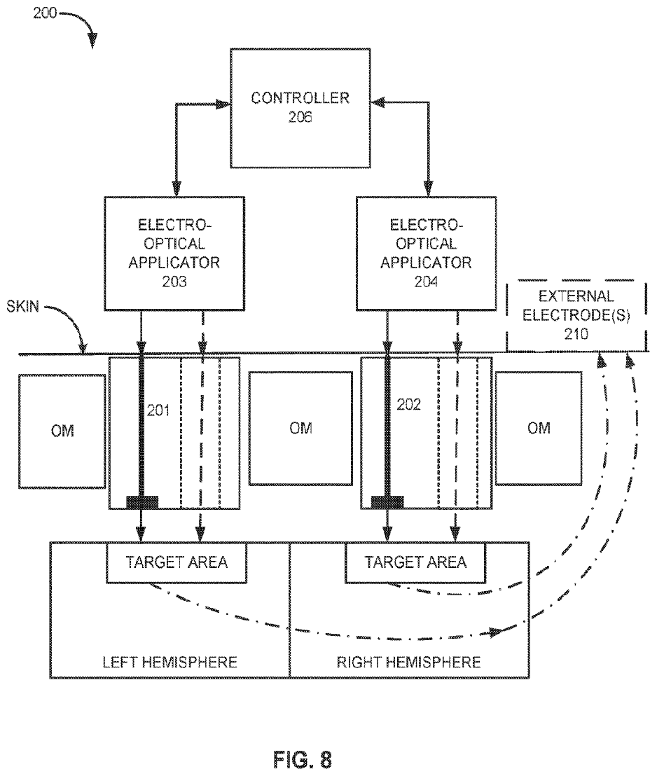

FIG. 8 shows an example configuration of system 200 where each of first and second electro-optical applicators 203 and 204 apply a light signal for PBM (the same and/or different, simultaneously and/or sequentially in time) through the windows 201 and 202 to the respective target areas. Additionally, each of the first and second electro-optical applicators 204 and 204 apply an electrical signal (the same and/or different, simultaneously and/or sequentially in time) through the windows 201 and 202 to the respective target areas. Each of the electrical signals can then be returned from the patient's tissues via one or more external electrode(s) 210 specifically positioned to make the current from the applied electrical signals flow through each of the target areas.

The currents can be subsequently returned to at least one of the electro-optical applicators 203, 204 and/or controller 206. Exact dosing schedules/schemes can be patient specific and/or based on the neurological disorder to be treated.

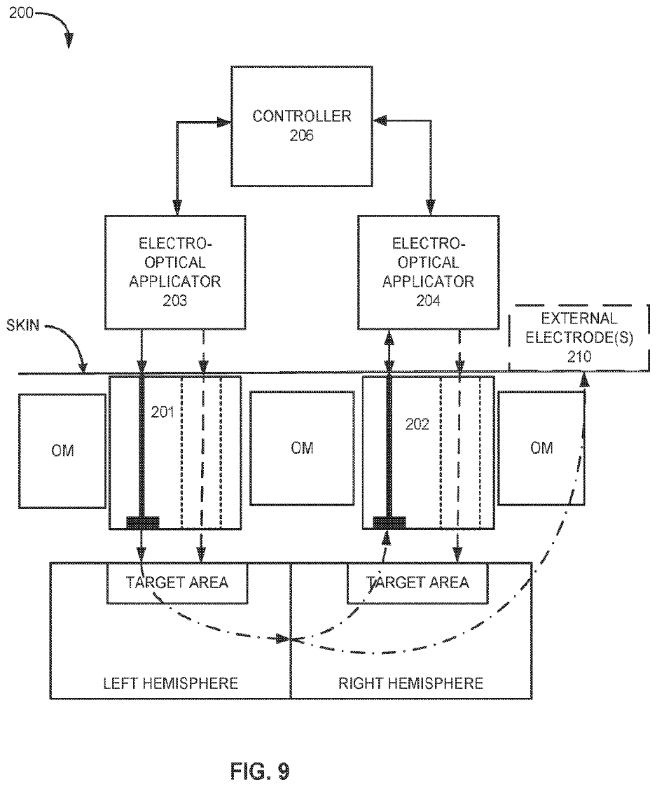

FIG. 9 shows additional examples of different configurations for signal applications utilizing system 200. Each of first and second electro-optical applicators 203 and 204 can apply a light signal for PBM (the same and/or different, simultaneously and/or sequentially in time) through the windows 201 and 202 to the respective target areas. The first electro-optical applicator 203 can apply an electrical signal through a window 201 to a target area in the respective hemisphere. In one instance, the current of the electrical signal can then pass through the first target area to the second target area to return through the electrode of the second window 202 It should be understood that charge current delivery in one direction is shown, but charge recovery in the opposite direction can also occur, for instance to avoid corrosion effects. While charge current delivery is shown in only one direction, it should be understood the reverse direction is possible (with charge recovery being the direction shown). In such a manner the current can be passed between the first and the second windows 201 and 202. In another instance, the current of the electrical signal can pass through at least the first target area to the one or more external electrodes 210 (and, not shown, can then be returned to the controller 206 and/or one of the electro-optical applicators 203, 204). In another instance, the current of the electrical signal can be passed through at least the first target area, and optionally the second target area, and returned partially though the second window 202 and partially though the one or more external electrodes 210. The different electrical signal applications can be combined with various PBM therapies for improved efficacy of treatments for one or more neurological disorders. It should be understood that the current need not flow as illustrated, and the illustrated current flow is for ease of drawing and understanding. For instance, current flow can be along a plurality of paths within the volume, while only one is shown. Further, it should be understood that each of the windows 201, 202 can behave similarly to the window 102 described with respect to FIGS. 1 and 2.

### IV. Methods

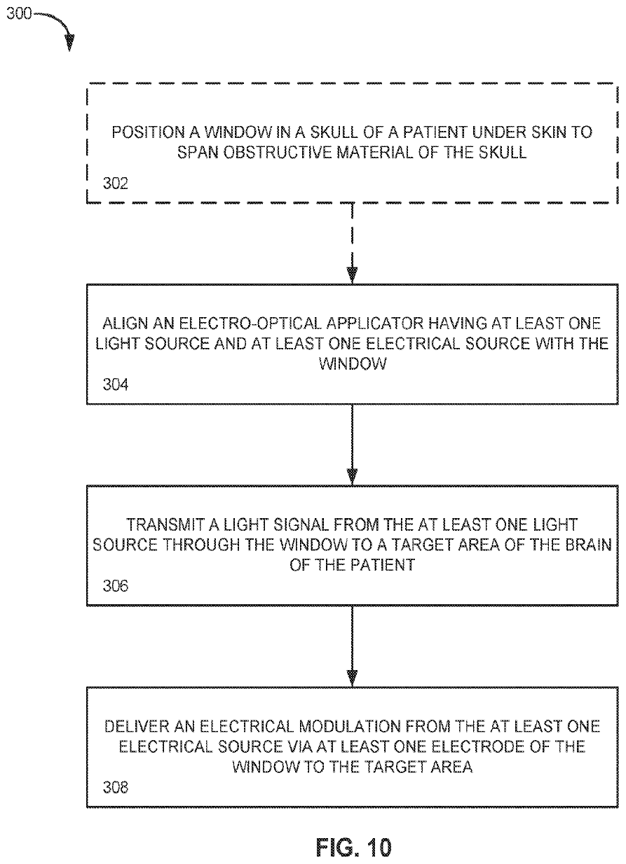

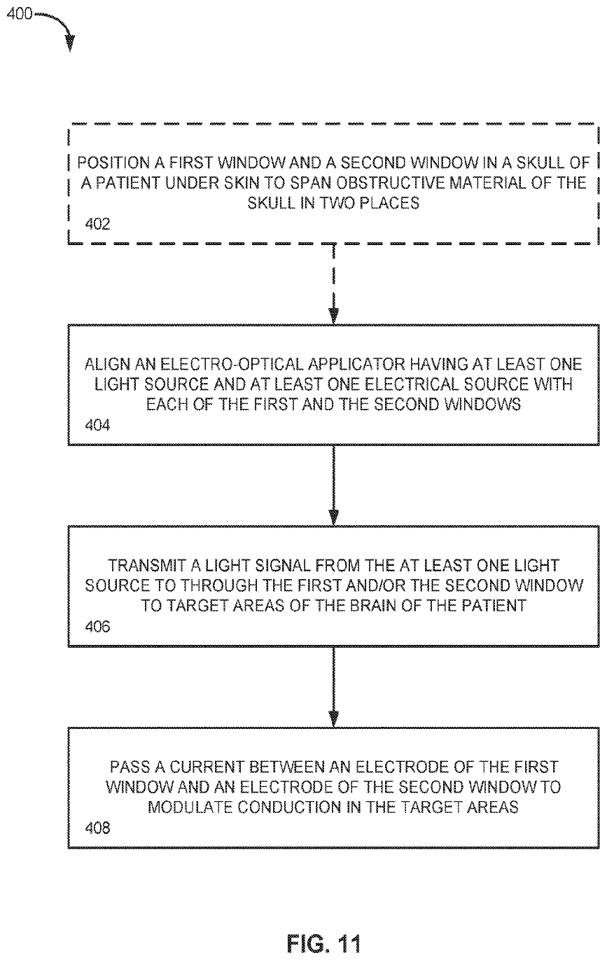

Another aspect of the present disclosure can include methods 300 and 400 (FIGS. 10 and 11) for delivering light signal(s) of photobiomodulation (PBM) and electrical stimulation transcutaneously from an external light source and external electrical source (e.g., electro-optical applicator 104) to a target area of a patient's brain via an electro-optical cranial window (also referred to as a window). Traditionally, a subcutaneous obstructive material (such as bone, dura, etc.) can hinder (e.g., by attenuating, refracting, resisting, etc.) a light signal delivered transcutaneously from reaching the target area and hinder an electrical signal (e.g., by insulating, etc.) being delivered to the target area. For example, the obstructive material can be bone of the skull which can attenuate the light signal of the PBM so much that the prescribed amount of light does not reach the target area, rendering the therapy less effective or ineffective. Electrical signals from skin surface electrodes can also be hindered to the point of ineffectiveness for deeper targets. Advantageously, the transcutaneous light and electrical delivery of the methods 300 and 400 is aided by a subcutaneously implanted window (e.g., window 102, and windows 201 and 202, aspects of which are shown in FIGS. 1-9) created in obstructing material between skin and the target area. Moreover, not requiring implantation of a light source and/or electrode and associated circuitry into brain material makes the system significantly less invasive. Keeping the majority and/or all of the electronics, controls, and power external to the patient's body can also increase safety, decrease costs, and allow for easier alteration of prescribed therapies while still delivering the prescribed therapies to the target area.

For purposes of simplicity, the methods 300 and 400 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the method, nor is the method necessarily limited to the illustrated aspects.

Referring now to FIG. 10, illustrated is a method 300 for delivering a light signal for PBM and/or electrical signal to a target area of a brain of a patient for a therapeutic effect on a neurological disorder. The target area of the patient's brain is obstructed by at least the skull, which can disrupt efficacious quantities of PBM and/or electrical signals when applied purely from above the skin. Optionally, at 302 a window can be positioned in the skull of the patient underneath the skin and spanning the obstructive material of the skull. In some instances, the window can be positioned in a pre-existing hole (e.g., from an accident, defect, or previous surgery) and/or, in other instances, surgically implanted. This step is optional because a window that has been previously implanted can be left within the obstructive material (without being removed) for a period of time (one or more hours to one or more years) that can allow a plurality of therapeutic sessions to occur and only needs to be positioned once (or re-positioned if necessary). The window can include at least one light transmission region and at least one electrical transmission region that can be positioned for sending light and/or electrical signals to the target area. It should be understood that a plurality of windows can be placed at different locations in the patient's body through different or the same obstructive materials in the patient's body (each window may be identifiable by different unique IDs, for example).

In one example, the window (e.g., window 102) can be implanted in a location under a patient's skin through at least a portion of obstructive material between the patient's skin and the target area. The window can be implanted without penetrating the target area but may contact the target area in some instances. The implantation can include, for example, making a hole in at least a portion of the obstructive material, using a hole already existing in a portion of the obstructive material, or widening/deepening an already existing hole. The implantation can include incising at least a portion of the skin of the patient. For instance, the window can be implanted so as to span through bone, dura, and/or muscle. Before use, the patient's skin may be allowed to heal over the window (e.g., window 102). As used herein, the term “over the window” can include an incision near, around, etc., the window. For example, a neurosurgeon can cut a patient's skin so to create a flap whose edges do not cross over the window, allowing the applicator/window to be used sooner without having to wait for the incision to heal and to avoid having to pass light through scar tissue generated during the healing process. The skin can heal over the window to lessen the chance of negative implications of the window's implantation (e.g., surgery related illnesses). As an example, features of the window, like the unique ID, can be stored with the information about the window to identify the window before any treatment is undertaken.

At 304, an electro-optical applicator including at least one light source and at least one electrical source can be aligned with the window. The alignment can be, for example, mechanical and/or magnetic. Optionally, the electro-optical applicator can include a safety feature to not provide an electrical signal and/or light signal unless the electro-optical applicator is properly aligned with the window (e.g., with one or more feedback loops). At 306, at least one light signal can be transmitted from the at least one light source of the electro-optical applicator through the light transmission region(s) of the window to the target area of the patient's brain. At 308, an electrical modulation made of at least one electrical signal from the at least one electrical source of the electro-optical applicator can be delivered to the target area via at least one electrode of the electrical transmission region of the window (through a conductive path). The at least one light signal and the at least one electrical signal can be applied to the target area simultaneously and/or separately according to a dosing scheme. The parameters of the light signal for the PBM, the parameters of the electrical signal, and/or at least a portion of the dosing scheme of the PBM and/or electrical signal can be configured by the controller (e.g., controller 106) and then sent to an electro-optical applicator (e.g., electro-optical applicator 104) to generate the light and/or electrical signals.

In some instances, the window can include at least one sensor and/or associated electronics for closed loop feedback and control of one or more parameters. A closed loop with the at least one sensor in and/or associated with the window can be used by a controller (e.g., controller 106 or 206), which defines parameters for the light delivered by the light source and the electrical signal delivered by the electrical source (e.g., of electro-optical applicator 104, or 203 and 204), to regulate the amount of light and/or current delivered by the electro-optical applicator (which, as previously noted, may be within the controller housing or external thereto). For example, the window (e.g., window 102) can include one or more components (e.g., electronics and/or sensors 126) that can facilitate a feedback signal being transferred to the controller (e.g., controller 106). Additionally and/or alternatively, the electro-optical applicator (e.g., electro-optical applicator 104) can include one or more sensor(s) that can measure at least one parameter and send a feedback signal to the controller. In some instances, the feedback can allow the controller (e.g., controller 106) to track the amount of light and/or current being received by the target area. In other instances, the feedback can provide the temperature of the window, the skin, and/or the target area. In each instance, the feedback can be used to adjust the parameters for the light and/or electrical signals to better match the prescribed amount of light/current and/or to keep the temperature within a predetermined threshold. It should be understood that the controller is described as working in a closed loop, the controller may also work in an open loop (user-in-the-loop) system.

Referring now to FIG. 11, illustrated is a method 400 for delivering at least one light signal for PBM and at least one electrical signal for electrical modulation to a target area in a brain of a patient utilizing two electro-optical cranial windows. The target areas of the patient's brain are obstructed by at least the skull (e.g., bone, dura, and the like), which can disrupt efficacious quantities of PBM and/or electrical signals when applied purely from above the skin. Optionally, at 402 a first window and a second window can be positioned in the skull of the patient underneath the skin and spanning the obstructive material of the skull in two places. The windows can be positioned in a pre-existing hole (e.g., from an accident, defect, or previous surgery) and/or surgically implanted in a newly created hole. This step is optional because the windows can be left within the obstructive material for a period of time (from 1 hour to several years) corresponding to plurality of therapeutic sessions and need only be positioned once (or re-positioned if necessary). Each of the windows can include at least one light transmission region and at least one electrical transmission region that can be positioned for sending light and/or electrical signals to the respective target area. It should be understood that a plurality of windows can be placed at different locations in the patient's body through different or the same obstructive materials in the patient's body (each window may be identifiable by different unique IDs, for example).

At 404, an electro-optical applicator including at least one light source and at least one electrical source can be aligned with each of the windows. The alignment can be, for example, mechanical and/or magnetic. Optionally, the electro-optical applicator can include a safety feature to not provide an electrical signal and/or light signal unless the electro-optical applicator is properly aligned with the window (e.g., with one or more feedback loops). Each of the windows can include a unique ID to inform the electro-optical applicator of the prescribed dosing scheme for that window. At 406, at least one light signal can be transmitted from the at least one light source of each of the electro-optical applicators (simultaneously and/or in a sequence or pattern) through the light transmission region(s) of the respective windows to the target areas of the patient's brain. At 408, a current can be passed between the first window and the second window (e.g., between an electrode of the first window and an electrode of the second window) to modulation conduction in at least one of the target areas of the brain. The electrical signal can be delivered from the at least one electrical source of one of the electro-optical applicators and can be delivered via at least one electrode of the electrical transmission region of one of the windows (through a conductive path). The current can then return through the second window, and optionally back through the second electro-optical applicator. The at least one light signal and the at least one electrical signal can be applied to at least one the target area simultaneously and/or separately, and from either window at a same or different time, according to a dosing scheme. The parameters of the light signal for the PBM, the parameters of the electrical signal, and/or at least a portion of the dosing scheme of the PBM and/or electrical signal can be configured by the controller and then sent to each of the electro-optical applicators to generate the light and/or electrical signals.

It should be noted that the light signal(s) for the PBM and the electrical signal(s) for electrical modulations discussed above can be applied at different target areas of the brain through either the same or different windows created through obstructive materials of the skull. One or more electro-optical applicators can be used for generating the light signal(s) and electrical signal(s). The electro-optical applicators(s) can be driven by a controller that can configure each PBM and electrical modulation according to a dosing scheme, user input, and/or closed loop control. Additionally, communication can be established between the controller and the window(s) in certain instances. For example, the communication can include feedback from a sensor of the window that the controller can use to estimate an amount of light and/or current being delivered to the target area in a dose, a heat of a target area, a total amount of light and/or current being delivered to the target area, alignment of the electro-optical applicator with the window, and/or determine the identity of the window based on a unique ID, or the like. As another example, the electro-optical applicator can include a temperature sensor configured to detect a temperature of the skin at and/or near the site(s) of light and/or electrical stimulation, and a temperature of the skin can be used as feedback to control the delivery of the light and/or the electrical signals. The controller, can for instance, determine if continuous or pulsed light should be delivered until the skin temperature reaches a certain level, then pause until the temperature returns to a lower threshold, then begin again. It should be noted that heat at the skin (e.g., sensed by a temperature sensor) can be used as a control-for example, light (constant and/or pulsed) can be delivered until the skin temperature reaches a certain level (e.g., a predefined high threshold) then the light can be paused until the temperature returns to a lower threshold (e.g., a predefined lower threshold) and the light can be administered again. As an example, at least two thresholds can be used for stability, but more thresholds can be used.

### V. Example Use

In this example use, one or more electro-optical cranial windows (also referred to as “windows”), as described above, can be placed in a patient's skull above the precuneus region of the brain (a node in the Default Mode Network (DMN)). Some data suggests that the DMN can be activated indirectly by DBS of other areas of the brain or peripheral nerve stimulation of other areas of the brain. In the past the DMN node of the precuneus has been targeted directly with transcranial magnetic stimulation (TMS) and shown favorable results. However, a challenge with TMS is that it must be applied in the clinic, so users only get therapy doses when they go to the clinic, which creates burdens for the health care system and may not enable patients to receive the number of treatments required for optimal therapy. Less invasive electrical and light based stimulation with a system that can be administered at home is ideal. Stimulating electrically through the cranium would be difficult and potentially dangerous. A fully implantable system is possible, but are quite expensive, more complex, and would limit availability to those that need it most and create other burdens on the healthcare system. The systems described herein implant a passive module (e.g., an electro-optical window) that enables electrical modulation and photobiomodulation access to the brain by passing through the intact skin and bypassing the cranium. The system can be powered by a take-home external module (e.g., electro-optical applicator) that would be easy to administer at home and is cost-reasonable.

The one or more windows and an associated external electro-optical applicator can deliver one or more of a light signal for photobiomodulation (PBM) and an electrical signal (e.g., current) for electrical stimulation/modulation (each configured according to a prescription). The one or more windows can span through the obstructive material (e.g., bone, dura, etc.) between the skin and the precuneus that can otherwise hinder transcutaneous application of light signals and/or electrical signals to portions of the brain. The one or more windows can transmit the light signal and the electrical signal received from the associated external electro-optical applicator to a target area in the precuneus region of the brain. As such, the external applicator can be configured to deliver both light and an electrical signal to the window.

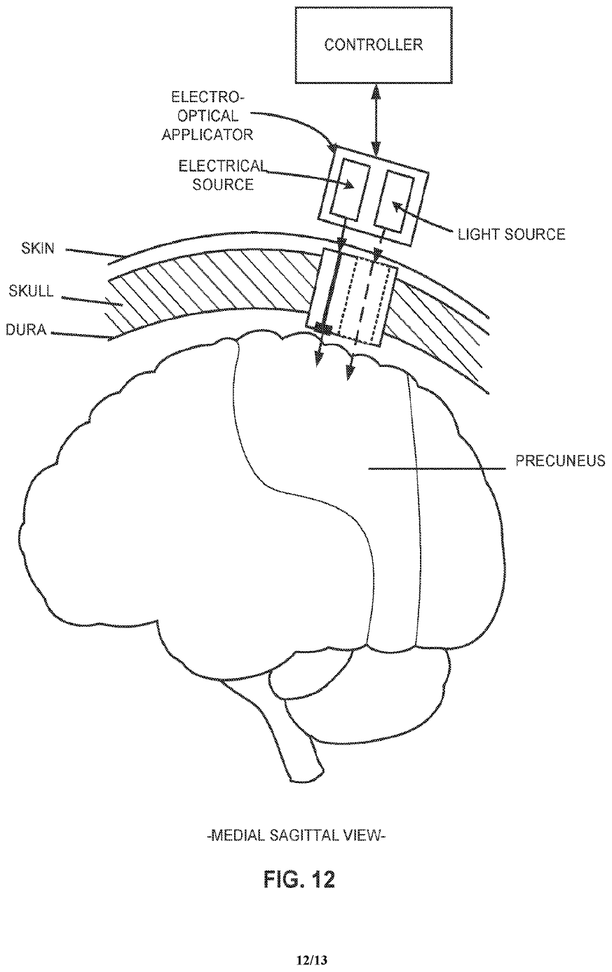

FIG. 12 shows a medial sagittal illustration of a portion of a patient's head with a single window positioned in the skull and configured to transmit light and an electrical signal (e.g., current) to a precuneus region of the brain. The window lies under the skin and spans through at least the bone of the skull and the dura (each significantly thicker than the skin and more obstructive to light and electrical signals than skin), as shown. The system shown in FIG. 12 is similar to the system 100 shown in FIG. 1 and/or one half of the system 200 shown in FIG. 7.

Referring to the one window illustrated in FIG. 12, the electro-optical applicator can be aligned to the window (e.g., with magnetic and/or mechanical alignment as described in detail above). The electro-optical applicator can include at least one electrical source and at least one light source. The electro-optical applicator can be in wired and/or wireless communication with a controller and/or power source for determining at least one parameter for the light signal and the electrical signal generated and applied by the electro-optical applicator. The light signal and the electrical signal, each with at least one parameter configured by the controller according to a dosing scheme and/or a prescription, can be generated by the electro-optical applicator and transmitted to the window. The light signal and the electrical signal can be transmitted through the window to the target area in the precuneus (and, in some instances, can have an effect throughout the DMN) or other cortical area. The light signal parameters for PBM can include power, duration, pulsatile or temporal delivery schemes, temperature management, whether or not stimulation and PBM are concurrent, and/or the like. Electrical signal parameters can include polarity, amplitude, pulse width, pulse timing/rate/pattern, selection of electrodes (e.g., within window and/or external electrodes in one or more external patches), impedance/continuity checks, and/or the like. The window can have a high optical transparency region (e.g., light pipe or the like) configured to deliver the light signal for PBM end an insulated electrically conductive region including a conductive path extending to an electrode at the bottom of the window near and/or adjacent to a portion of the precuneus. It should be understood that one or more light signals and electrical signals can be applied simultaneously and/or in a predetermined pattern for therapeutic effect. For instance, applying light signal(s) and electrical signal(s) to the precuneus can at least partially treat dementia or another neurological disorder linked with the DMN.

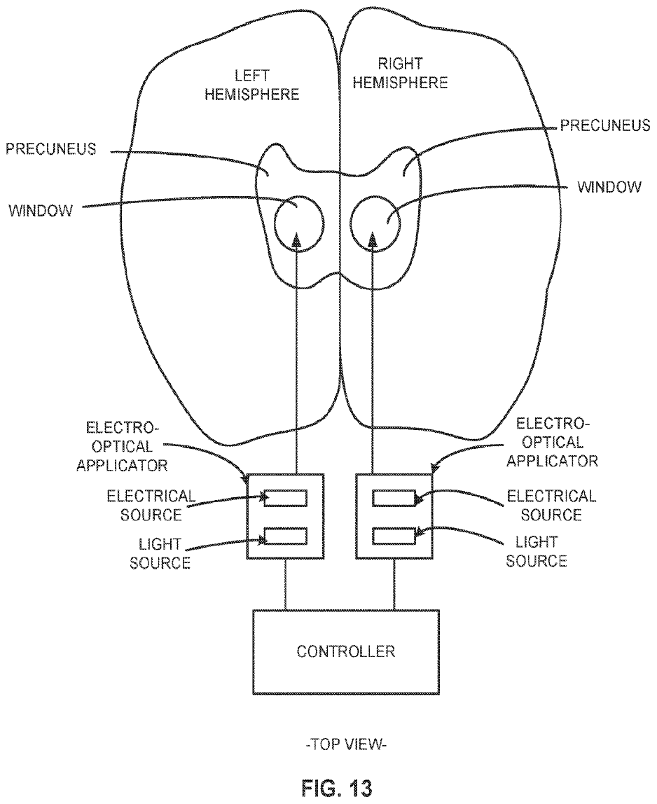

FIG. 13 shows a top (transverse) view of a patient's brain (skull not shown) with two windows positioned above the precuneus region of each hemisphere of the brain. The two windows are configured to transmit light signal(s) and electrical signal(s) (e.g., current) to the associated precuneus region of the brain. Current can be passed between the two windows and/or with one or more external electrode(s) (not shown), to electrically modulate the target areas in the precuneus (which may also affect other portions of the DMN). The windows described herein are similar to those described with respect to FIG. 7 and can pass current in any of the ways described with respect to FIGS. 8 and 9. Two electro-optical applicators connected to a controller are illustrated, but it should be understood that a single applicator unit may be used and moved between the two windows or can include two or more electrical and light sources. It should also be understood that more than two windows may exist (e.g., four windows, sixteen windows, thirty-two windows, or more). The light signal and the electrical signal can work together to treat one or more neurological disorders, including Alzheimer's disease, autism, schizophrenia, major depressive disorder (MDD), chronic pain, post-traumatic stress disorder (PTSD), attention deficit hyperactivity disorder (ADHD), and the like that may be associated with a disruption in the DMN. For instance, PBM and electrical modulation can be applied to the precuneus to at least partially treat dementia. It should be noted that although the precuneus region is described in this example use, other brain areas may be considered and found to be preferable.

From the above description, those skilled in the art will perceive improvements, changes, and modifications. Such improvements, changes and modifications are within the skill of one in the art and are intended to be covered by the appended claims.

1. A system comprising:
at least one electro-optical applicator external to a patient, each comprising:
at least one light source, and
at least one electrical source; and
at least one window spanning beneath skin covering a skull of the patient and through obstructive material of the skull of the patient, each comprising at least one electrode proximal a target area of a brain of the patient and a conductive path to the at least one electrode, wherein the at least one window is configured to:
transmit a light signal from the at least one light source through the window to the target area of the brain of the patient; and
deliver an electrical modulation from the at least one electrical source via the at least one electrode to the target area of the brain of the patient.

2. The system of claim 1, wherein the at least one window spans through the obstructive material, creating a path for light transmission from outside the skull of the patient to the target area of the brain of the patient.

3. The system of claim 1, wherein the target area of the brain is within a default mode network.

4. The system of claim 1, wherein the target area of the brain is at least a portion of the precuneus.

5. The system of claim 1, wherein the at least one electro-optical applicator is configured to align the at least one light source with the at least one window to transmit the light signal.

6. The system of claim 1, wherein the at least one electro-optical applicator and the at least one window each comprise a magnet and/or ferromagnetic material that facilitates the alignment.

7. The system of claim 1, wherein the window comprises a high optical transparency region for transmitting the light signal through the obstructive material, wherein the conductive path and/or the at least one electrode is positioned on the at least one window so not to interfere with the light signal.

8. The system of claim 1, wherein at least a portion of the at least one electrode is positioned at and/or near a bottom of the window near the target area of the brain of the patient.

9. The system of claim 1, further comprising a controller coupled to the at least one electro-optical applicator and configured to set at least one light signal parameter and/or at least one electrical modulation parameter and control application of the light signal and/or the electrical modulation.

10. The system of claim 9, wherein the at least one electrical modulation parameter comprises at least one of a polarity, an amplitude, a pulse width, a pulse timing, a pulse rate, a pulse pattern, and/or selection of one or more of the at least one electrode.

11. The system of claim 9, wherein the controller is further configured to perform an impedance and/or a continuity check of the system.

12. The system of claim 9, wherein the at least one light signal parameter comprises at least one of a power, a duration, a pulsatile delivery scheme, a temporal delivery scheme, a wavelength, and/or a timing of light signal delivery.

13. The system of claim 9, wherein the controller is further configured to regulate a timing of the electrical signal in concert with a timing of the light signal delivery.

14. The system of claim 9, wherein the delivery of the electrical stimulation is concurrent with transmission of the light signal and/or the delivery of the electrical stimulation is separate from the transmission of the light signal.

15. The system of claim 9, further comprising a temperature sensor configured to sense a temperature of the target area and/or the skin, and the controller is further configured to determine the at least one light signal parameter based on a pre-determined temperature management limit for the target area and/or the skin.

14. (canceled)

15. (canceled)

16. The system of claim 1, wherein the light signal and the electrical stimulation are delivered to treat dementia, Alzheimer's disease, autism, schizophrenia, major depressive disorder, chronic pain, and/or post-traumatic stress disorder.

17. A system comprising:
a first window configured to span through obstructive material of a skull of a patient between the skin and a precuneus of a hemisphere of a brain of the patient and comprising:
a first electrode proximal the precuneus of the hemisphere to deliver an electrical stimulation, and
a conductive pathway from a top of the window to the first electrode;
a first electro-optical applicator comprising a light source and an electrical source and configured to align the light source with the first window to deliver a light signal through the first window to the precuneus of the hemisphere and the electrical source with the conductive pathway;
a second window configured to span through obstructive material of the skull of the patient between the skin and another portion of the precuneus of the precuneus of the hemisphere or a portion of another precious of another hemisphere of the brain of the patient and comprising:
a second electrode proximal the precuneus of the hemisphere to deliver an electrical stimulation, and
another conductive pathway from a top of the window to the first electrode; and
a second electro-optical applicator comprising another light source and another electrical source configured to align the other light source with the second window to deliver another light signal through the second window to the other portion of the precuneus or the other portion of the precuneus of the other hemisphere and the other electrical source with the other conductive pathway.

18. The system of claim 17, further comprising a controller coupled to the first and the second electro-optical applicators and configured to set at least one light signal parameter for the first light signal and another at least one light signal parameter for the second light signal and at least one electrical stimulation parameter for the first electrode and another at least one electrical stimulation parameter for the second electrode.

19. The system of claim 17, wherein a current is passed between the first and the second electrodes.

20. The system of claim 19, further comprising at least one external electrode coupled to the controller, wherein a current is passed between the at least one external electrode and the first electrode and/or the second electrode.
